(19) United States
(12) Patent Application Publication
(10) Pub. No.: US 2013/0072917 A1
(43) Pub. Date: Mar. 21, 2013

(54) DEVICE AND METHOD FOR CATARACT SURGERY

(71) Applicants: Michael Kaschke, Oberkochen (DE); Ludwin Monz, Jena (DE); Tobias Damm, Munchen (DE); Dirk Mühlhoff, Jena (DE); Karlheinz Rein, Aalen (DE); Gregor Stobrawa, Jena (DE); Manfred Dick, Gefell (DE); Martin Kühner, Bad Klosterlausnitz (DE); Artur Högele, Oberkochen (DE); Matthias Reich, Jena (DE); Dieter Grebner, Grosslobichau (DE)

(72) Inventors: Michael Kaschke, Oberkochen (DE); Ludwin Monz, Jena (DE); Tobias Damm, Munchen (DE); Dirk Mühlhoff, Jena (DE); Karlheinz Rein, Aalen (DE); Gregor Stobrawa, Jena (DE); Manfred Dick, Gefell (DE); Martin Kühner, Bad Klosterlausnitz (DE); Artur Högele, Oberkochen (DE); Matthias Reich, Jena (DE); Dieter Grebner, Grosslobichau (DE)

(73) Assignee: CARL ZEISS MEDITEC AG, Jena (DE)

(21) Appl. No.: 13/700,396

(22) PCT Filed: May 26, 2011
(86) PCT No.: PCT/EP2011/002608

§ 371 Date: Nov. 27, 2012

(30) Foreign Application Priority Data
May 27, 2010 (DE) 102010022298.4
May 27, 2010 (US) 61349042

(51) Int. Cl. A61F 9/008

(52) U.S. Cl. CPC A61F 9/008
USPC 606/6

(57) ABSTRACT

Improvements in respect of performing cataract surgery, and the result thereof, by application of a laser system. A device for cataract surgery, includes a surgical microscope or stereo microscope and a laser source. A module, consisting of a laser-coupling/deflecting unit, a laser-scan unit, and a focusing unit, can be attached to the surgical microscope or stereo microscope, in which at least one of these units can selectively be introduced between the surgical microscope and eye, and in which the focusing unit can scan a depth-of-focus range of greater than 1 mm.

### PRIORITY CLAIM

The present application is a National Phase entry of PCT Application No. PCT/EP2011/002608, filed May 26, 2011, which claims priority from German Application No 10 2010 022 298.4, filed May 27, 2010, and U.S. Provisional Application Ser. No. 61/349,042, filed May 27, 2010, the disclosures of which are hereby incorporated by reference herein in their entirety.

### FIELD OF THE INVENTION

The invention relates to improvements in respect of performing cataract surgery, and the result thereof, by application of a laser system.

### BACKGROUND

To date, the following steps are typically performed on the eye during cataract surgery, which eye has been dilated by drops (i.e. the pupil has been dilated by medicaments) and is under local anesthetic:
Making an approximately 1.5-3 mm wide cut, set manually, into the cornea using a scalpel to provide access to the anterior chamber of the eye for all that follows and
making a circular cut, set manually, with a diameter of approximately 3-6 mm into the anterior capsular bag using a special scalpel; removing the capsular-bag segment in order to provide access to the lens.
Cutting apart the lens or the lens fragments, and subsequent further subdivision thereof, combined with suctioning off the fragments using an ultrasound device/rinsing-suctioning device using different ultrasound energy levels and rinsing speeds or suction pressure levels (phacoemulsification).
Inserting an intraocular lens into the capsular bag.

As an alternative to the previously used instruments (surgical microscope, phacoemulsification apparatus, also referred to as phaco machine), or in addition thereto, use is made of a femtosecond laser system (fs-laser) for:
cutting into the cornea,
cutting into the capsular bag,
cutting apart and further subdividing of the lens,
making optionally desired or necessary relaxing cuts into the cornea (in order to compensate for astigmatism).

The use of a cutting laser makes it possible to perform more precisely positioned cuts, which are more defined in terms of their dimensions. As a result of the additional implementation of a navigation, e.g. by means of optical coherence tomography in conjunction with the laser system, the processes of cutting and dividing can be automated, without tissue worth retaining, e.g. the posterior capsular bag, being injured. As a result, the significant risks of manual/visual cut guidance by the operator are avoided and even medical practitioners with less surgical experience achieve a lower complication rate and better refractive results.

WO09039302 describes such a laser system: a laser is guided onto the eye via a deflection mirror and an objective. An x/y/z scanner moves this laser beam in the eye and performs cuts. The z-scan can also be embodied as a displacement of the objective along the optical axis. There, OCT (optical coherence tomography) is used for navigation. An fs-laser or a ps-laser without more detailed specification is mentioned as a laser. The laser system is designed as an independent system, with the imaging required for the navigation being integrated therein. It operates independently from phaco machines and surgical microscopes. This allows the laser cuts to be able to be performed before suctioning off the lens fragments and before inserting the lens, e.g. in a different spatial area. However, this assumes a modified process organization in the hospital or the surgical practice. Alternatively, the laser system must be pushed to-and-fro in an intricate and laborious fashion in a conventionally organized operating room. An expensive fs-laser is necessary for the implementation; the aperture is restricted as a result of the large working distance, and this leads to a majority of the residual energy of the laser being radiated in the direction of the retina and constituting a safety risk.

A different laser system is described in U.S. Pat. No. 7,621,637, which is designed only for refractive corneal surgery. It is a system in which a laser is swiveled into the surgical region between the microscope lens of the surgical microscope and the eye by use of a swivel arm. A horizontal flap-cut plane is made in the cornea by a slow movement of the objective along one direction and a rapid movement of a tilting mirror, which is housed in an independent module. The advantage of this system lies in the simple integration into the corneal-surgery procedure. By way of example, the observation by the operator is only interrupted during the swiveling-in and during the flap cut.

The use of an expensive fs-laser and the long scan time by the objective scan were found to be disadvantageous, although the latter is acceptable during a corneal-flap cut because only one cut needs to be made. Furthermore, as a result of the principles thereof, the depth of focus in this system is not deep enough to be able to perform cuts in the lens, and it lacks a 3D navigation, which is necessary for lens cuts. Moreover, the overall device is too voluminous for the typical operation situation (surgical microscope, phaco machine).

### SUMMARY OF THE INVENTION

It is an object of the invention to develop a laser system for cataract surgery, which has a more compact design and can therefore be integrated more easily into the existing system surroundings, is more cost-effective but nevertheless meets the requirements of laser-lens surgery, such as large aperture (in order not to burden the retina and in order to obtain a sufficiently small focus) and high scan speed (in order to be able to perform all necessary steps, more particularly for comminuting the lens, within approximately 1 minute).

In a first variant (A1), this object is achieved by a laser- and navigation system as an add-on module for a surgical microscope, including:
1. a femtosecond (fs)-laser or a picosecond (ps)-laser;
2. a) a module, which can be added on, with a high-aperture objective with a large adjustable range of the focus, which module is held at the microscope such that it can swivel and can be positioned in a defined fashion between microscope objective and contact lens or eye,
b) or, alternatively, a module, which can be added on, with a high-aperture objective, which module is held fixedly at the microscope and can be positioned in a defined fashion between microscope objective and contact lens or eye, wherein the module additionally has a lens for partial compensation of the objective effect when looking through the objective;
3. optionally a contact lens (such that the overall system, made of the objective and, optionally, the contact lens, has a numerical aperture (NA)>0.2);
4. a deflection/diffraction unit, which couples the laser into the objective, as a component of the module. In the case 2.b), it is embodied as a dichroic mirror (transmits in the visible range, reflects in the laser-wavelength range);
5. two fast scanners with a mirroring embodiment or a fast 2-axes scanner, which deflect(s) the laser beam in the x/y direction, or a scanner and elements for beam rotation as a component of the module;
6. optionally a detection unit, which detects the laser light reflected/scattered from the eye and obtains navigation data therefrom for the online control and the pre-orientation of the cut pattern;
7. a laser feed, preferably an optical waveguide or a free-beam articulated arm, which feeds the laser beam to the module from the laser source.

This variant is particularly suitable for a large number of cuts when comminuting the lens.

In an alternative variant (A2), this object is achieved by a laser- and navigation system with add-on modules for a surgical microscope, including:
1. an fs-laser or a ps-laser;
2. a module, which can be added on, with a high-aperture objective with a large adjustable range of the focus, which module is held at the microscope such that it can swivel and can be positioned in a defined fashion between microscope objective and contact lens or eye;
3. optionally a contact lens (such that the overall system, made of the objective and, optionally, the contact lens, has a NA>0.2);
4. a unit that couples the laser into the microscope, wherein the microscope optical system couples the laser into the displaceable high-aperture-like objective;
5. a beam scanner, consisting of e.g. two fast scanners with a mirroring embodiment or a fast 2-axes scanner, which deflect(s) the laser beam in the x/y direction, or a scanner and elements for beam rotation as a component of the objective module, which can be added on, or as a separate module in the laser beam feed to the surgical microscope;
6. optionally a detection unit, which detects the laser light reflected/scattered from the eye and obtains navigation data therefrom for the online control and the pre-orientation of the cut pattern.

This variant is likewise particularly suitable for a large number of cuts when comminuting the lens, and wherein the microscope already contains coupling-in means for a laser, e.g. also via the optical interface of a co-observation optical system.

In a further alternative variant (B1), this object is achieved by a laser- and navigation system as an add-on module for a surgical microscope, including:
1. an fs-laser or a ps-laser;
2. a module, which can be added on, with a high-aperture objective that can be displaced in the x/y/z-direction within the module, which module is held at the microscope such that it can swivel and can be positioned in a defined fashion between microscope objective and contact lens or eye;
3. optionally a contact lens (such that the overall system, made of the objective and, optionally, the contact lens, has a NA>0.2);
4. a unit that couples the laser into the microscope, as a component of the module;
5. a 3-axes objective positioning unit, implemented by means of e.g. piezo-drives or stepper- or servo motors;
6. optionally a detection unit, which detects the laser light reflected from the eye and obtains navigation data therefrom for the online control and the pre-orientation of the cut pattern;
7. a laser feed, preferably an optical waveguide or a free-beam articulated arm, which feeds the laser beam to the module from the source.

This variant is particularly suitable for a relatively small number of cuts, e.g. if only a cross cut of the lens is intended to be cut, or else if only tough, dense cataract regions are intended to be cut.

In a further variant (B2), this object is achieved by a laser- and navigation system with add-on modules for a surgical microscope, including:
1. an fs-laser or a ps-laser;
2. a module, which can be added on, with a high-aperture objective that can be displaced in the x/y/z-direction within the module, which module is held at the microscope such that it can swivel and can be positioned in a defined fashion between microscope objective and contact lens or eye;
3. optionally a contact lens (such that the overall system, made of the objective and, optionally, the contact lens, has a NA>0.2);
4. a unit that couples the laser into the microscope, wherein the microscope optical system couples the laser into the displaceable high-aperture objective;
5. a 3-axes/3D objective positioning unit, implemented by means of e.g. piezo-drives or stepper- or servo motors. As an alternative to the x/y or x-scan, the objective can also be tilted by motor in the x/y axes or in the x-axis;
6. optionally a detection unit as an add-on for the microscope or the module, which can be added on, which detection unit detects the laser light reflected from the eye and obtains navigation data therefrom for the online control and the pre-orientation of the cut pattern;
7. a laser feed, preferably an optical waveguide or a free-beam articulated arm, which feeds the laser beam to the microscope from the source.

This variant is particularly suitable for a relatively small number of cuts, e.g. if only a cross cut of the lens is intended to be cut, or else if only tough, dense cataract regions are intended to be cut and when the surgical microscope already contains coupling-in means for a laser, e.g. also via the optical interface of a co-observation optical system.

Here, the fs-laser preferably has a pulse duration of between 100 fs and 1000 fs, with a pulse energy of between 0.10 μJ and 10.00 μJ and repetition frequencies of between 50 kHz and 500 kHz. What this energy and these pulse durations achieve is that the laser can produce a vapor/plasma bubble in a tissue volume with a diameter of approximately 5 micrometers, with only small effects being induced outside of this volume. The high repetition frequency, in conjunction with the fast scanners, affords the possibility of, within one minute, being able to produce at least 8 vertical/or radial and 2 horizontal cuts in the lens, which has a thickness of between 3-6 mm.

The ps-laser preferably has a pulse duration of between 1 ps and 20 ps, with a pulse energy of between 1 μJ and 200 μJ and repetition frequencies of between 25 kHz and 150 kHz. What this energy and these pulse durations achieve is that the laser produces a vapor/plasma bubble in a tissue volume with a diameter of approximately 10-15 micrometers. However, unlike the fs-laser, the ps-laser affects a larger volume on account of the thermal effects and pressure effects. However, this is largely uncritical, seeing as the lens is in any case removed entirely during the cataract operation and the cuts to the cornea and in the capsular bag need not satisfy the precision (e.g. in respect of coarseness, dimensional accuracy) required for optical imaging. However, the pulse duration and energy should not lie substantially above the specified values because otherwise there is a significant increase in the risk of injury to the corneal endothelial cells by pressure peaks or to the anterior capsular bag segments remaining in the eye after the intervention.

What holds true for both lasers is that in the case of relatively small lenses or if the cuts are only performed in the dense regions of the lens or if the cuts only serve to replace the initial ultrasound cross cut, fewer cuts than the aforementioned approximately 8 are also expedient, and so the repetition frequency or the overall time for the cuts may be reduced.

However, in the case of a dense or tough cataract or cataract regions—identifiable e.g. from the stray-light data from the detector unit—the number of cuts overall may also be increased, or else there may be a local increase in the cut density only in the dense or tough cataract regions. In particular, the distance between 2 cut surfaces can be reduced to the typical geometry/dimension of the suction-head inlet, or to less than that. As a result, the fragments of the lens need not be comminuted much more by a subsequent phaco step. This can significantly reduce the ultrasound energy or, ideally, this may allow the use of ultrasound to be dispensed with entirely.

The invention furthermore includes a method for laser-assisted eye surgery, in which the laser-beam source is flexibly connected to a scanning and focusing module, which, mechanically balanced in direct contact with the eye, is used for intraocular navigation and therapy.

### BRIEF DESCRIPTION OF THE DRAWINGS

The invention will be explained in more detail in the following text on the basis of the drawings, in which:

FIG. 1 is a schematic illustration of the invention according to variant A1,

FIG. 2 is a schematic illustration of the invention according to variant A1 in a 2^{nd} embodiment,

FIG. 3 is a schematic illustration of the invention according to variant A2,

FIG. 4 is a schematic illustration of the invention according to variant A2 in a 2^{nd} embodiment,

FIG. 5 is a schematic illustration of the invention according to variant B1, and

FIG. 6 is a schematic illustration of the invention according to variant B2.

### DETAILED DESCRIPTION

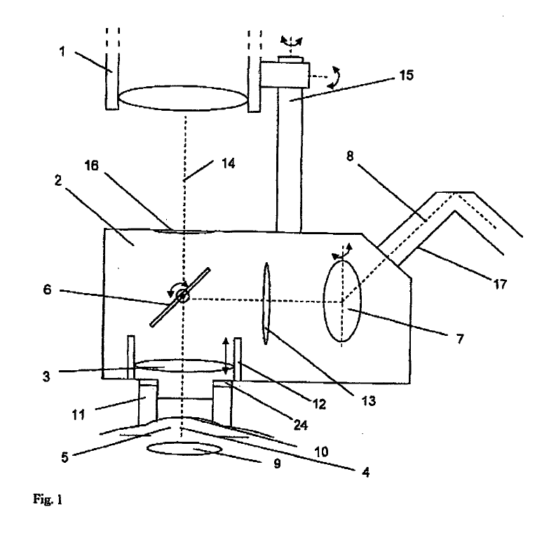

FIG. 1 illustrates the invention according to variant A1. It includes the module 2, which can be added on to a surgical or stereo microscope 1, an objective 3 with great displacement of the focus along the optical axis 4 of the eye 5, and a deflection unit 6, 7 for coupling in the laser beam 8, wherein the deflection unit 6, 7 is embodied as a scanning unit for deflecting the laser in the x/y-direction.

In one focusing position, the objective 3 is able to focus onto the rear side of the lens 9 of the eye and, in another focusing position, said objective is able to focus onto the front side of the lens 9 of the eye; advantageously it is additionally also able to focus onto the front side of the cornea 10. The focus drive 12 serves to shift the position of the focus. At the same time, the objective 3 must be able to cover a scanning field with the diameter of a pupil dilated by drops or from the center of the pupil to the sclera. It must likewise have an aperture large enough to ensure that the light is sufficiently defocused on the retina so that no injury threshold of the retina is exceeded by the light cone during a treatment duration of approximately 1 minute. That is to say the through-focusing region is greater than 10-12 mm, at least greater than 1 mm, in a field with a diameter of greater than 4 mm and an aperture of greater than approximately 0.20. In order to achieve this in the case of a small overall size and low weight, aspherical or free-form lens surfaces and/or diffractive elements and/or a contact lens 12 (with planar contact surface, or a contact surface matched to the corneal curvature) and/or adaptive mirror surfaces may be used. The contact lens 11 is affixed on the eye by means of negative pressure.

By way of example, two fast galvo-scanners are options for the scanners 6, 7. However, a scanner that scans along the meridian and is itself mechanically rotated, or the beam of which is rotated, by e.g. a prism, (so that there is a meridian-rotation) is also an alternative option. As a further alternative option, use can also be made of a MEMS scanner that can move along 2 axes. In the embodiment illustrated in FIG. 1, the objective 3 of the module 2 is in the imaging beam path of both the laser (not illustrated here) and the microscope 1. An advantage of this is that the aperture of the objective 3 can be selected to be very large; a relay lens 13 serves for matching the laser beam 8 to the observation beam path 14 of the microscope 1.

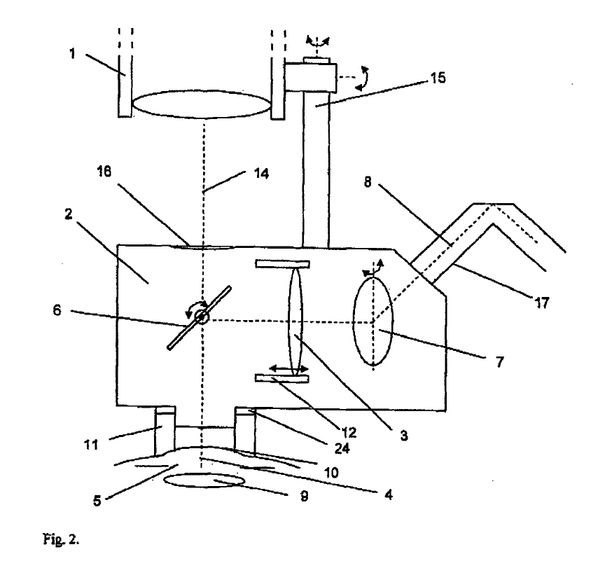

However, the objective 3 may also be installed in the beam path of the laser, upstream of where said beam path merges into the observation beam path 14 of the microscope 1, e.g. between the two scan mirrors 6, 7, as illustrated in FIG. 2. This affords the possibility of better independent regulation of the foci of laser and observation beam path 14.

A fastener 15 connects the module 2 to the microscope 1 e.g. such that it can swivel. The module furthermore has an entry window 16 for the observation beam path 14, which entry window may also be embodied as a matching lens. The laser beam 8 is coupled into the module 2 via a feed 17, which may be embodied as a fiber or else as a free-beam apparatus.

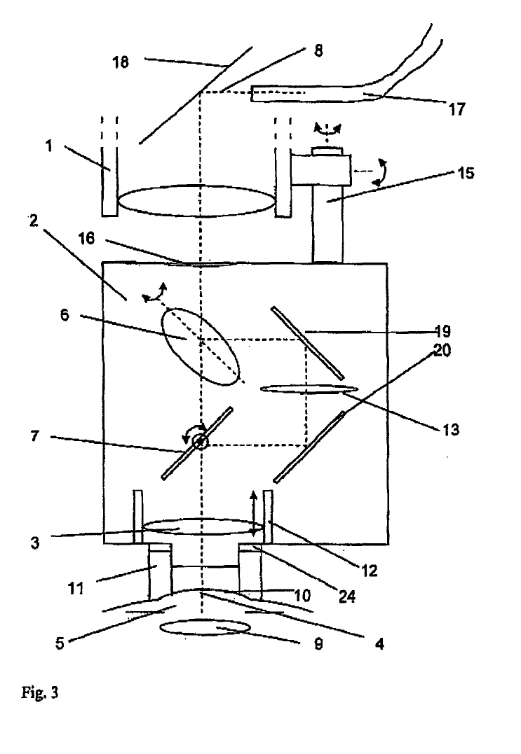

In variant A2, the laser is firstly coupled into the microscope, and the latter transmits the laser into the objective 3. This is illustrated in FIG. 3. Hence the microscope 1 itself has the deflection or beam splitter unit 18. Scan elements 6, 7 for laser-beam scanning can be positioned upstream of where the laser is coupled into the microscope or, as illustrated, between the microscope 1 and the objective 3. The scan apparatus moreover has two additional fixed reflectors 19, 20 and a relay lens 13. Otherwise, identical elements in FIG. 3 have identical reference signs as in FIGS. 1 and 2; reference is made to the description relating to these.

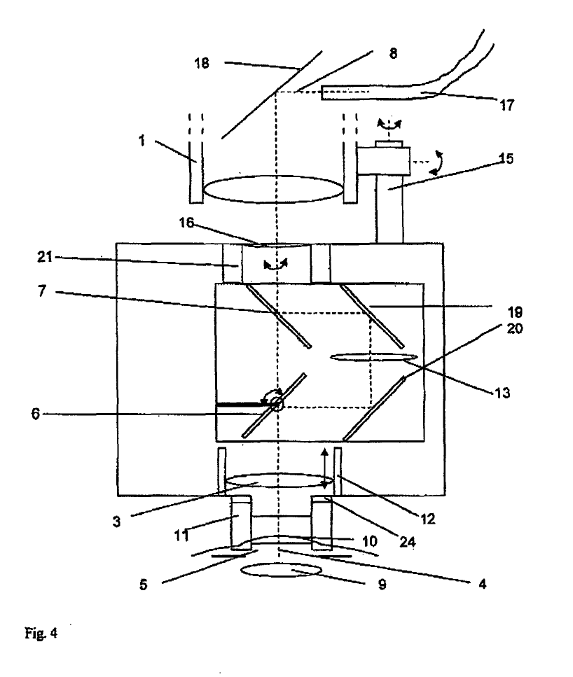

FIG. 4 shows a further embodiment of the variant A2. Here, use is made of only one movable deflection element 6; deflection element 7 is fixed. The required second movement direction of the scanned laser beam 8 is implemented by rotation of the entire module 2 about the optical axis 4 by means of the circular guide 21. The position of the deflection elements 6, 7 may also be interchanged with the position of the reflectors 19, 20 in both FIG. 3 and FIG. 4.

In variants B1 and B2, the module 2, which can be added on to a surgical or stereo microscope 1, contains an objective 3 with mechanical displacement of the focus both along the optical axis of the eye and laterally in the x/y-direction. Here the same conditions for the necessary adjustment tracks of the focus of the objective 3 hold true as in the variants A1 and A2.

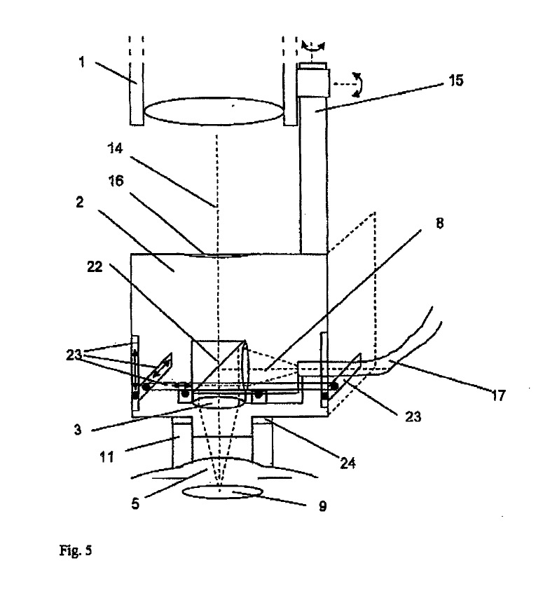

FIG. 5 illustrates an embodiment of the module 2 according to variant B1. Here, the laser beam 8 is coupled into the module 2 via a deflection unit 22 (as in FIGS. 1 and 2). The three-dimensional movement of the focus of the laser beam 8 is implemented by an x/y/z-movement of the objective 3, e.g. along guides 23 by means of piezo-elements or stepper- or servo motors, with or without position feedback.

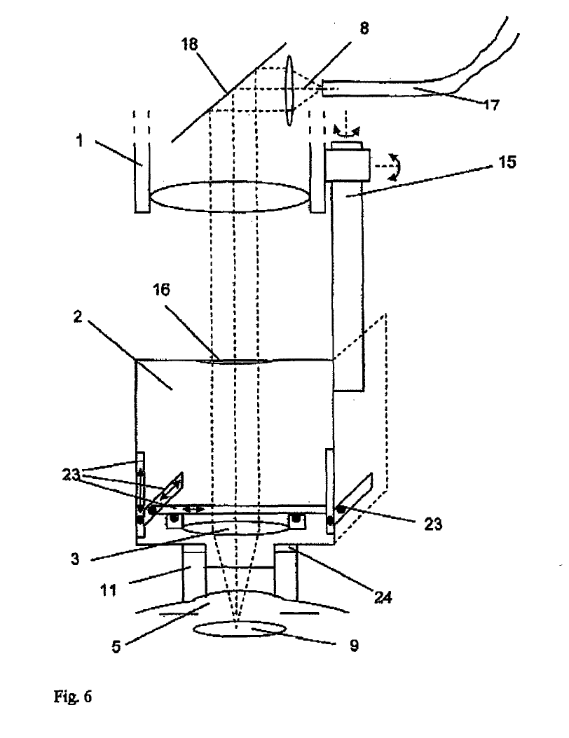

FIG. 6 shows an embodiment according to variant B2. Here, the laser beam 8 is firstly coupled into the microscope 1, and from there it is coupled into the objective 3 of the module 2. As in FIG. 5, the focus adjustment is implemented by 3-dimensional movement of the objective 3 by means of the guides 23. Unlike variant B1, the beam cone in the object, e.g. the lens of the eye, impinges obliquely on the object for x/y focus positions away from the optical axis of the eye and is therefore subject to less interference by the iris.

A confocal detector (not illustrated here) or a planar detector serves as a detection unit. The confocal signal serves for determining the boundaries, as is described in DE 103 23 422, the entire content of which is incorporated by reference. Together with the non-confocal component, this allows a scattering intensity to be determined, and this scattering intensity can be used to control the laser in terms of one or even more of the following parameters: pulse energy, pulse duration, repetition frequency and/or scan speed. The reflected light may optionally be decoupled via a combination of wave plate and polarization splitter. An OCT unit may also be considered as detection unit.

So that the overall module 2 is made manageable from a mechanical standpoint for an operator, the structural elements in the module 2 are arranged and distributed such that the center of gravity of the module 2 is situated below the objective of the surgical microscope head 1, but it is at least situated along the nadir from the center of gravity of said microscope head.

Furthermore, a device 24 for generating a minimum contact pressure of the module 2 on the contact lens 11 is integrated into the module 2. This can be implemented by a pressure transducer, e.g. by a spring or an electromechanical pressure actuator, which, from the module, presses against the contact lens or the eye with a defined force, the latter optionally being fed-back via a sensor. This device can also be able to move (pressure-distance transducer) the contact lens 11 in the direction of the eye over the small distances (1 mm).

In order to aid integration and simplify the operation, provision is made—integrated into the module or provided externally—for a controller/control unit that supports the following operational procedure:
1. The operator optionally (a) places the contact lens on the eye, which has been dilated by drops and is anesthetized, or, alternatively, (b) the contact lens is placed onto the add-on module.
2. The operator moves the microscope head over the contact lens or eye. The lateral position and the position relating to the distance (size of the centering) from the contact lens or the eye can be set by said operator by use of a centering, which is reflected in or identified by use of a monitor image. The module is swiveled in manually or automatically once a suitable distance is established; in order to establish the contact between the module and contact lens in case (a), the microscope head is displaced to the eye and/or the pressure-distance transducer is lengthened, until there is the necessary contact with the eye and the necessary contact pressure thereon. Thus suction pressure is applied during this or thereafter. In order to be able to establish the contact between the module/contact lens and eye in case (b), the microscope or the pressure-distance transducer can likewise be utilized in a fashion that is analogous to case (a).
3. The laser system scans the eye area in 3D at low energy levels. The detection unit determines landmarks and the envisaged cut pattern is oriented on the real 3D structure. The cut spacing in accordance with the cataract density is optionally obtained from the detector data and modified such that the remaining fragments can be destroyed at low ultrasound energy and can be suctioned in.
4. The laser cuts apart the lens from posterior to anterior on the basis of the oriented cut pattern and under online navigation/laser-parameter monitoring.
5. The laser cuts apart the capsular bag.
6. The laser makes the corneal cut, optionally after a preceding repeated 3D orientation.
7. Contact pressure and suction pressure are removed. The microscope and/or the pressure-distance transducer are pulled away from the eye. The module is swiveled away manually or automatically. The contact lens is removed manually.
8. Optionally, information relating to the position of the cuts, particularly in the cornea and in the capsular bag, is displayed on the monitor image or reflected into the OPMI.
9. The operator manually continues the cataract surgery.

The invention is not restricted to the illustrated exemplary embodiments; developments by a person skilled in the art do not depart from the scope of protection.

1-15. (canceled)

16. A device for cataract surgery, comprising:
a surgical microscope head or stereo microscope head and a laser source;
a module, including a laser-coupling/deflecting unit, a laser-scan unit, and a focusing unit, the module being removably attachable to the surgical microscope or stereo microscope;
wherein at least one of the laser-coupling/deflecting unit, the laser-scan unit, and the focusing unit is selectively interposable between surgical microscope and an eye; and
wherein the focusing unit scans a depth-of-focus range of greater than 1 mm.

17. The device as claimed in claim 16, in which the focusing unit comprises at least one aspherical surface, at least one diffractive element or an adaptive element.

18. The device as claimed in claim 16, in which the module has a first center of gravity and the surgical microscope head or stereo microscope head has a second center of gravity and the first center of gravity of the module when attached to the surgical microscope head or stereo microscope head is situated below the second center of gravity of the microscope head or coincides therewith or is situated along the nadir from the center of gravity of the microscope head.

19. The device as claimed in claim 16, in which two reflecting scanners, or a scanner and a rotation element, or a two-axis scanner, or the objective are integrated in the module in elements that can be displaced laterally in two dimensions.

20. The device as claimed in claim 19, in which one of the two reflecting scanners, the scanner and the rotation element or the two axis scanner is simultaneously embodied as deflection unit.

21. The device as claimed in claim 16, wherein the laser source comprises a fs-laser, with a pulse duration of 100-1000 fs, or a ps-laser, with pulse duration of 1-20 ps.

22. The device as claimed in claim 16, further comprising a detection unit that detects reflected light, which has been reflected in a confocal and/or planar fashion, and utilizes said light for pre-orientation of a desired cut pattern, a navigation or laser-parameter control.

23. The device as claimed in claim 22, wherein the detection unit is integrated into the module.

24. The device as claimed in claim 22, in which the reflected light and current data obtained therefrom are compared to data from a biometry obtained preoperatively.

25. The device as claimed claim 16, further comprising a contact lens that is used in addition to the objective.

26. The device as claimed claim 16, further comprising an adjustment device that positions the surgical microscope head or the stereo microscope head and the module over the contact lens or the eye the adjustment device, the adjustment device being operably coupled to the microscope and/or the module.

27. The device as claimed in claim 16, further comprising contact-pressure and/or suction devices or contact-pressure and/or suction pressure transmission lines operably coupled onto or into the module.

28. A method for laser-assisted cataract surgery, in which a cut or shot distance at least in some lens regions is smaller than or equal to the suction opening of a suction device.

29. A method for laser-assisted cataract surgery, comprising making a cut or shot distance, at least in some lens regions smaller than or equal to a suction opening of a suction device.

30. A method for laser-assisted eye surgery, in which a laser-beam source is flexibly connected to a scanning and focusing module, which, mechanically balanced in direct contact with the eye, is used for intraocular navigation and therapy.

31. A method for laser-assisted eye surgery, comprising:
flexibly connecting a laser-beam source to a scanning and focusing module;
mechanically balancing the scanning and focusing module in direct contact with the eye; and
using the scanning and focusing module for intraocular navigation and therapy.

32. A device for cataract surgery, comprising:
a surgical microscope head or stereo microscope head and a laser source;
a module, including a laser-scan unit, and a focusing unit, the module being removable attachable to the surgical microscope or stereo microscope;
wherein at least one of the laser-scan unit, and the focusing unit is selectively interposable between surgical microscope and an eye;
wherein the microscope includes a laser-coupling/deflecting unit and the microscope couples the laser into the focusing unit, and
wherein the focusing unit scans a depth-of-focus range of greater than 1 mm.

33. The device as claimed in claim 32, in which the focusing unit comprises at least one aspherical surface, at least one diffractive element or an adaptive element.

34. The device as claimed in claim 32, in which the module has a first center of gravity and the surgical microscope head or stereo microscope head has a second center of gravity and the first center of gravity of the module when attached to the surgical microscope head or stereo microscope head is situated below the second center of gravity of the microscope head or coincides therewith or is situated along the nadir from the center of gravity of the microscope head.

35. The device as claimed in claim 32, in which two reflecting scanners, or a scanner and a rotation element, or a two-axis scanner, or the objective are integrated in the module in elements that can be displaced laterally in two dimensions.

36. The device as claimed in claim 35, in which one of the two reflecting scanners, the scanner and the rotation element or the two axis scanner is simultaneously embodied as deflection unit.

37. The device as claimed in claim 32, wherein the laser source comprises a fs-laser, with a pulse duration of 100-1000 fs, or a ps-laser, with pulse duration of 1-20 ps.

38. The device as claimed in claim 32, further comprising a detection unit that detects reflected light, which has been reflected in a confocal and/or planar fashion, and utilizes said light for pre-orientation of a desired cut pattern, a navigation or laser-parameter control.

39. The device as claimed in claim 38, wherein the detection unit is integrated into the module.

40. The device as claimed in claim 38, in which the reflected light and current data obtained therefrom are compared to data from a biometry obtained preoperatively.

41. The device as claimed claim 32, further comprising a contact lens that is used in addition to the objective.

42. The device as claimed claim 32, further comprising an adjustment device that positions the surgical microscope head or the stereo microscope head and the module over the contact lens or the eye the adjustment device, the adjustment device being operably coupled to the microscope and/or the module.

43. The device as claimed in claim 32, further comprising contact-pressure and/or suction devices or contact-pressure and/or suction pressure transmission lines operably coupled onto or into the module.
